(19) United States
(12) Patent Application Publication
(10) Pub. No.: US 2006/0024378 A1
(43) Pub. Date: Feb. 2, 2006

(54) Magnetically responsive carbon nano-structures for transporting biologically active substances, and methods relating thereto

(76) Inventor: Rakesh H. Mehta, Ashburn, VA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/040,426

(22) Filed: Jan. 21, 2005

Related U.S. Application Data
(60) Provisional application No. 60/598,023, filed on Aug. 2, 2004.

(51) Int. Cl. H01F 1/00; A61K 9/14

(52) U.S. Cl. 424/489; 252/62.51 R

(57) ABSTRACT

This invention is directed to a composition for medicinal transportation in a biological fluid. The carrier composition is a single or multi-walled nanostructure, open or closed at either end, having a non-metallic ferromagnetic component (A), a metallic ferromagnetic component (B), and a carbon component (C), where the atomic ratio of A:B:C is: 0.1-200: 0.05-75: 100. The cross-sectional size of the nanostructures of the present invention is less than 30 nanometers in at least one direction.

This application claims the benefit of U.S. Provisional Application 60/598023, filed Aug. 2, 2004.

### FIELD OF THE INVENTION

The present invention relates generally to compositions and methods for delivering biologically active agents to a selected location in a living organism. More specifically, the present invention is directed to magnetically responsive carbon nanostructures as medicinal carriers to provide local influence on pathological structures in the body.

### BACKGROUND OF THE INVENTION

U.S. Pat. No. 5,651,989 to Volkonsky, et al., describes carbon based carrier compositions (used in the treatment of various disorders), guided or controlled by external application of a magnetic field. Carbon based carrier compositions can however: i. lack adequate capacity for transporting the desired biologically active agent to the treatment site; ii. have less than desirable magnetic susceptibility; and/or iii. be difficult to manufacture, store and/or use (e.g., require an unduly high flux density magnetic field for controlling movement; require unduly complex adjustments to the magnetic field with each new carrier material, due to magnetic property variability; and/or require complex sterilization procedures).

### SUMMARY OF THE INVENTION

The present invention is directed to a magnetically responsive carrier composition for medicinal transportation in a biological fluid. The carrier composition is a single or multi-walled nanostructure, open or closed at either end, having a non-metallic ferromagnetic component (A), a metallic ferromagnetic component (B), and a carbon component (C), where the atomic ratio of A:B:C is:
for component A, an amount within a range between and including any two of the following 0.1, 0.5, 1, 2, 3, 5, 8, 10, 15, 18, 20, 25, 30, 40, 50, 60, 75, 100 and 200;
for component B, an amount within a range between and including any two of the following: 10⁻⁴, 10⁻³, 0.05, 0.1, 0.2, 0.5, 0.6, 0.7, 0.8, 0.9, 1, 2, 3, 4, 5 10, 11, 12, 13, 14, 15, 16, 17, 18, 19, 20, 21, 22, 23, 24, 25, 30, 40, 50, 60, and 75; and

for component C, an amount of 100,

The cross-sectional size of the nanostructures of the present invention is less than 30, 25, 20, 15, 12, 10, 9, 8, 7, 6, 5, 4, or 3 nanometers in at least one direction.

### DETAILED DESCRIPTION OF THE PREFERRED EMBODIMENT(S)

The present invention is directed to the use of a magnetically responsive, carbon nanostructure. “Carbon nanostructure” is intended to mean any nano-scale, (primarily, if not exclusively sp² type) carbon structure, such as, a carbon nano-tube, a carbon nano rope, a carbon nano-horn, a fullerene, a graphene sheet and/or derivations and/or combinations thereof, including derivatives therof which are primarily, if not exclusively, sp² molecular structures, only partially (if at all) comprising carbon, such as sp² molecular structures comprising: boron, aluminum, gallium, indium, silicon, germanium, tin, lead, nitrogen, phosphorus, arsenic, antimony, oxygen, sulfur, selenium, tellurium, zinc, cadmium, and combinations thereof. The carbon nano-structures of the present invention are created from at least three gas streams:

i. a carbon containing stream for building the carbon-carbon structure, such as: a. vaporized graphite or other inorganic having relatively large amounts of carbon (e.g., greater than 80, 90, 95, 98 or 99 weigh percent carbon moieties); or b. a substituted or unsubstituted organic gas, particularly low molecular weigh organic gases (e.g., having a molecular weight of less than 100, 80, 60, 50 or 40) with alkene or alkyne functionality;

ii. a vaporized metal which provides catalytic type assistance to the reactions necessary for creating the carbon structure, while portions of the metal also become incorporated into or onto the carbon structure, such as, cobalt, copper, nickel, iron, nickel, zinc, palladium, and silver; and

iii. a dopant gas for incorporating relatively small amounts of non-metalic constituents into or onto the carbon-carbon structure, where the dopant differs in valence electrons from carbon, and is able to cause an unbalanced difference in electron density (which in turn provides ferromagnetism), such as phosphorous, nitrogen, or boron.

The carbon nanostructures of the present invention are (at least partially) made ferromagnetic by the non-metallic dopant (such as nitrogen, boron or phosphorous). The metal component further adds ferromagnetic properties. The metallic and non-metallic ferromagnetic components of the present invention can be fine-tuned, depending upon the particular chosen application, to have optimal ferromagnetic and low toxicity properties (metals are often more difficult to metabolize and may present a higher health hazard than, for example, nitrogen).

Furthermore, non-metallic ferromagnetic components tend to be less dense (a better match to the density of blood or other body fluids) and thereby provide a better support for efficiently and effectively carrying medicinal agents.

In one embodiment, the only requirement for the non-metallic species is that it differs in valence electrons from carbon and is able to wholly or partially displace carbon during the formation of the nanostructure, thereby causing an unbalanced difference in electron density (which in turn provides ferromagnetism).

The carbon nanostructure generally comprises an atomic ratio of A:B:C, where: A (representing the non-metallic species) is a range between and including any two of the following 0.1, 0.5, 1, 2, 3, 5, 8, 10, 15, 18, 20, 25, 30, 40, 50, 60, 75, 100 and 200, although 0.1 to about 30 is often most preferred, depending upon the particular application chosen; where B (representing the metallic component) is a range between and including any two of the following: 0.0001, 0.0005, 0.001, 0.005, 0.01, 0.05, 0.1, 0.2, 0.5, 0.6, 0.7, 0.8, 0.9, 1, 2, 3, 4, 5 10, 11, 12, 13, 14, 15, 16, 17, 18, 19, 20, 21, 22, 23, 24, 25, 30, 40, 50, 60, and 75, although 0.1 to about 30 is often most preferred, depending upon the particular application chosen; and where C (representing the carbon component) is 100. The nanostructure can be a single walled structure or multi-walled structure. The nanostructure can be open at one end or entirely closed.

Another embodiment of the present invention is a carbon nanotube that has some of its constituent carbon atoms replaced by doped nitrogen atoms and further comprises a metal. Carbon nanotubes are nanostructures having a cylindrical shape composed of a graphite layers. It is referred to as a single walled carbon nano-structure (SWCNT) if there is one graphite layer. Multi walled carbon nanotubes (MWCNT) can comprise, two or three or more graphite layer walls. Optionally, SWCNT and/or MWCNT of the present invention can have their ends covered with a semispherical cap composed of five-membered rings (otherwise known as a “fullerene cap”). In one embodiment, the nanotube is capped with a metal or metal complex.

A nitrogen-doped, metalized carbon nanotube nanostructure can be obtained by allowing a mixture gas of C₂H₂, N₂, and cobalt to flow by chemical vapor deposition (CVD) method under the following condition (given as a hypothetical example).
Flow rate of C₂H₂: 15 cubic centimeters per minute (ccpm)
Flow rate of N₂: 50 ccpm
Flow rate of cobalt vapor: 0.1 ccpm
Temperature: 1250° C.
   Alternatively, it may also be obtained by DC magnetron sputtering (or laser ablation) with a graphite (doped with cobalt) target in a mixed gas of argon and nitrogen.

The magnetically responsive nanostructures of the present invention have a very high surface area and readily adsorb soluted biologically active substances, such as, alkylating agents, antimetabolites, antitumor antibiotic chemotherapy agents or combinations thereof, and other therapeutic agents and drugs such as systemic toxicity inhibitors, hydracortosone or the like.

The ferromagnetic properties of the nano-structures of the present invention can be used to transport biologically active substances, where the transport can be modified by a magnetic field. The nanostructures tend to agglomerate, but such agglomeration should be controlled, if possible, since less agglomerated nanostructures transport biologically active ingredients generally faster and more efficiently. It is believed that the non-metallic ferromagnetic component is less prone to agglomeration and can therefore be advantageous in controlling the agglomeration of the nanostructures, through a fine tuning of the amount of non-metallic ferromagnetic component incorporated into the nanostructure.

Furthermore, smaller carbon nanostructures are generally easier to metabolize out of the body after transportation is complete (any restraining magnetic field is removed and the nanostructure is allowed to be metabolized, such as, in the liver). Preferred nanostructures (agglomerated or otherwise) have a particle size less than (or equal to) one of the following (in microns): 5, 4, 3, 2, 1, 0.9, 0.8, 0.7, 0.6, 0.5, 0.4, 0.3, 0.2, 0.1, 0.05, 0.02, 0.01, 0.005, 0.002, although with particle sizes exceeding 4.0 microns, an undesired degree of embolization of vessels becomes increasingly possible, unwanted coagulation of dispersion may take place (which makes injections more difficult) and the speed of discharging biologically active substances from the particles in the targeted pathological zones can be slowed. The nanostructures of the present invention are advantageous, due to their uniquely high surface area (per unit of weight), thereby allowing relatively high medicinal loadings relative to known carrier materials, resulting in smaller carrier particles often capable of carrying larger amounts of medicine. The compositions of the present invention can be utilized for localized in vivo treatment of disease. For example, the laden (with biologically active material) carrier can be injected into the body of a patient, by inserting (by hypodermic needle or other delivery means) in a blood vessel to within a short distance from a site to be treated and at a branch or branches (preferably the most immediate) to a network of vessels carrying blood at the site and injecting the carrier through the delivery means, and establishing a magnetic field exterior to the body and adjacent to the site of sufficient field strength to guide a substantial active substances and methods of production and use thereof.

When ready for use (or, in the alternative, before packaging where a carrier is to be delivered with a preselected biologically active substance already absorbed thereon), it is believed that greater than 200, 300, 400, 500, 750, or 1000 milligrams of the biologically active substance in solution can be added to 1 gram of the nanostructure carrier of the present invention. When ready for application to a patient, the combination can be placed into suspension (for example, 5 to 10 ml) utilizing normal procedures.

It is theorized that a magnetic field less than 250, 225, 200, 175, or 150 oersteds/cm may be sufficient to guide the nanostructures of the present invention after injection into a bodily fluid, depending upon the size of the nanostructure and amount of dopant and non-metallic ferromagnetic component incorporated into the nanostructure.

It is believed that under the influence of the applied magnetic field, the carrier particles can be induced into the capillary network feeding a tumor. The particles can be drawn closely adjacent to the soft tissue of the lumen of the capillaries (or perhaps even into the soft tissue) thereby reducing or eliminating the potential for embolization of the vessels. The biologically active substance can then be released from the carrier particles by a dynamic process of replacement of the substance in the carrier by materials produced by the body (for example the necrotic products of the tumor itself), such as proteins, glucose, lipids, peptides, or the like, thus literally pushing the biologically active substance off of the carrier.

The term “associated with” as used herein means that carrier can be coated, impregnated, or otherwise operably associated with a biological substance using techniques available to those skilled in the art. Examples of such techniques include adsorption, covalent attachment of the biological substance to the carbonaceous surface either directly or indirectly through the use of a suitable linking moiety, calcium precipitation, etc. DNA precipitation is described in Fitzpatrick-McElligott, Bio/Technology, 10(9): 1036-1040 (September 1992).

Examples of biological substances which can be associated with the nanostructures of the present invention include, but are not limited to, nucleic acids, genetic constructs, proteins such as enzymes, toxins, pharmaceutical compounds, viruses, hormones, lipids, biological stains, organelles, and vesicles. Preferably, the genetic construct should code for a protein with effective flanking regulatory sequences to express the protein in the target. It is also possible to use a genetic construct which is an RNA strand or DNA sequence effective to inhibit a native gene or to retard a disease process. DNA or RNA sequences and their derivatives which inhibit gene expression can also be referred to as antisense.

The compositions of the present invention (a carrier having a substantially pure carbonaceous surface to which is associated a biological substance) can be inserted into a target using any number of means available to those skilled in the art. There can be mentioned directed parenteral injection such as intramuscular, intravenous and subcutaneous. There can also be mentioned nasal sprays and implants as well as microinjection.

To replace the biologically active substance in the carrier particles, it is felt that the replacing substance must have a higher specific gravity than the biologically active substance, so it is advantageous to have a low density carrier material, such as the nanostructures of the present invention.

As may be appreciated, an improved magnetically responsive carrier for biologically active substances and methods for producing and using the same are provided by this invention, particles forming the carrier exhibiting improved responsiveness to magnetic fields, having improved absorptive capacity, and being durable during storage and use.

While the invention has been described in its preferred embodiments, it is to be understood that the words which have been used are words of description rather than limitation and that changes within the purview of the appended claims may be made without departing from the true scope and spirit of the invention in its broader aspects.

What is claimed is:

1. A magnetically responsive carrier composition for medicinal transportation in a biological fluid, comprising: a single or multiwalled nanostructure, open or closed at either end, having a non-metallic ferromagnetic component (A), a metallic ferromagnetic component (B), and a carbon component (C), where the atomic ratio of A:B:C is:
for component A, an amount within a range between and including any two of the following 0.1, 0.5, 1, 2, 3, 5, 8, 10, 15, 18, 20, 25, 30, 40, 50, 60, 75, 100 and 200;
for component B, an amount within a range between and including any two of the following: 0.0001, 0.0005, 0.001, 0.005, 0.01, 0.05, 0.1, 0.2, 0.5, 0.6, 0.7, 0.8, 0.9, 1, 2, 3, 4, 5 10, 11, 12, 13, 14, 15, 16, 17, 18, 19, 20, 21, 22, 23, 24, 25, 30, 40, 50, 60, and 75;
for component C, an amount of 100,
wherein the cross-sectional size of the nanostructure is less than 30 nanometers in at least one direction.

2. A composition in accordance with claim 1, wherein at least 10 weight percent of the nanostructure is a single walled carbon nanotube or a single wall carbon nanohorn, wherein said nanotube or nanohorn defines an average diameter of less than 5 nanometers.

3. A composition in accordance with claim 1 wherein at least 30, 40, 50, 60, 70, 80, 90 or 95 weight percent of the metallic ferromagnetic component (B) is cobalt.

4. A composition in accordance with claim 1 wherein at least 30, 40, 50, 60, 70, 80, 90 or 95 weight percent of the non-metallic ferromagnetic component (A) is nitrogen.

5. A composition in accordance with claim 1 further comprising a medicinal agent.

6. A composition in accordance with claim 1, wherein the metallic ferromagnetic component (B) and the non-metallic ferromagnetic component (A) are together present in an amount sufficient to cause the composition to be physically manipulated in a fluid by a magnetic field in a range between and including any two of the following: 250, 225, 200, 175, and 150 oersteds/cm.
